(19) United States
(12) Patent Application Publication
(10) Pub. No.: US 2005/0158812 A1
(43) Pub. Date: Jul. 21, 2005

(54) Kits and methods for autoantibody detection

(76) Inventors: Elizabeth A. Gomez, Chanhassen, MN (US); Curtis L. Shellum, Rochester, MN (US)

Correspondence Address: BECKMAN COULTER, INC., P.O. BOX 169015, MAIL CODE 32-A02, MIAMI, FL, 33116-9015, US

(21) Appl. No.: 10/759,411

(22) Filed: Jan. 16, 2004

(51) Int. Cl. G01N 33/543

(52) U.S. Cl. 435/7.93; 435/7.1; 436/501; 436/518

(57) ABSTRACT

An immunoassay method and test kit for detecting autoantibody in a patient sample, such as autoantibodies that interferes with the complexation of intrinsic factor with vitamin B12. A labeled receptor, i.e., intrinsic factor, is mixed with the sample and then with an anti-intrinsic factor antibody that binds competitively with the autoantibody to intrinsic factor bound to a solid phase is added and presence of autoantibody in the sample is determined.

### FIELD OF THE INVENTION

The invention relates to a device and method for detection of autoantibodies in a sample. In particular, the invention relates to a device and method for detection of intrinsic factor autoantibodies that block vitamin B12 binding to intrinsic factor using an immunoassay.

### BACKGROUND OF THE INVENTION

Autoimmune diseases arise when an immune system fails to recognize one or more of the body's components as its own and forms autoantibodies against that component. Various receptor substances are present in living organisms to which various molecules or proteins in the organism must bind for normal biological interactions to occur. When autoantibodies are formed that specifically bind to the receptor substance at a site that blocks the binding of the molecule the autoantibodies are referred to as “blocking autoantibodies.”

One common autoimmune disease of this type is pernicious anemia. Pernicious anemia results when vitamin B₁₂ (B₁₂) is not adequately absorbed by the body. In a normal patient, B₁₂ enters the gastrointestinal tract and combines with R-protein (also known as cobalophilin, haptocorrin, and transcobalamin), which picks up the B12 and transports it through the stomach and into the small intestine. The stomach cells also produce intrinsic factor, which travels to the small intestine. When the corrinoid-R-protein complex gets to the small intestine, the corrinoid is liberated from the R-protein by enzymes made by the pancreas. Of the liberated corrinoids, only the cobalamins attach to intrinsic factor. Intrinsic factor then carries the cobalamins to the last section of the small intestine, the ileum. B₁₂ cannot be absorbed by the body unless it is combined with intrinsic factor. B₁₂ binds to a specific epitope on intrinsic factor. This binding triggers a conformational change in intrinsic factor that allows the intrinsic factor-B₁₂ combination to be absorbed by the body.

In certain cases, a person's immune system mistakenly considers intrinsic factor to be a foreign substance and produces antibodies against intrinsic factor (anti-intrinsic factor antibodies). One type of such anti-intrinsic factor antibodies blocks the B₁₂ binding site on intrinsic factor, preventing B₁₂ from binding to the intrinsic factor. As a result, B₁₂ is not adequately absorbed by the body resulting in pernicious anemia.

Methods have been developed for the detection of particular autoantibodies, as the detection serves as a tool for diagnosing an autoimmune disease that results from the presence of that particular autoantibody.

Immunoassays have been used to detect and/or quantitate the presence of blocking autoantibodies. Typically, a radioimmunoassay (RIA) is used to detect the presence of such autoantibodies. The RIAs used to detect anti-intrinsic factor antibodies are typical of such assays. In RIAs currently in use to detect anti-intrinsic factor antibodies, intrinsic factor is bound to a solid phase. B₁₂ is labeled with a radioactive isotope such as cobalt 57, resulting in a cobalt 57-B₁₂ tracer. A patient sample is first mixed with the solid phase. During this step, patient anti-intrinsic factor antibodies bind to the intrinsic factor on the solid phase. The cobalt 57-B₁₂ tracer is then mixed with the patient sample and solid phase. The cobalt 57-B₁₂ tracer binds to intrinsic factor on the solid phase to which no autoantibody is bound. Washing is performed to wash away all substances not bound to the solid phase. The radioactivity from the tracer bound to the intrinsic factor on the solid phase is detected and the amount of radioactivity is inversely proportional to the amount of patient anti-intrinsic factor antibodies present in the sample.

One drawback with this RIA is that B₁₂ present in the patient sample may erroneously elevate the test results. High levels of B₁₂ may be present in patients who are undergoing B₁₂ injections for B₁₂ deficiency. B₁₂ binds to the intrinsic factor on the solid phase, reducing the amount of intrinsic factor available to bind to anti-intrinsic factor antibodies and/or tracer. Non-labeled B₁₂ that binds to the intrinsic factor-solid survives washing and thus is not detected. The level of radioactivity detected is decreased, which erroneously elevates the amount of anti-intrinsic factor antibody believed to be present in the patient sample.

A similar drawback to the RIA is that B₁₂ binding proteins present in a patient sample may also erroneously elevate the test results. B₁₂ binding proteins bind to the cobalt 57-B₁₂ tracer, which decreases the amount of tracer binding to intrinsic factor remaining on the solid phase. Since less tracer remains on the solid phase after washing, the level of radioactivity detected is decreased, which erroneously elevates the amount of anti-intrinsic factor antibody believed to be present in the patient sample.

While these known methods have provided researchers with various techniques to determine the presence of autoantibodies, these techniques have several disadvantages as described above. An immunoassay method for detecting blocking autoantibodies in a fluid sample that is sensitive and capable of being partially or fully automated is desired.

### SUMMARY OF THE INVENTION

The present invention provides a method for determining in a fluid sample the presence of autoantibodies that specifically bind to a binding site of a receptor in a living organism. In one aspect, the method comprises mixing the fluid sample suspected of containing the autoantibody with a labeled receptor having a specific binding site for said autoantibody. The receptor is any protein or binding factor present in a living organism having binding sites to which autoantibodies may bind and either block the binding of a molecule or protein that naturally binds to the receptor or that when bound stimulates the release of a hormone resulting in excess levels of such hormone in the body. Examples of receptors include intrinsic factor, thyroid stimulating hormone, folate stimulating factor, insulin receptors and the like.

After the labeled receptor is allowed to bind autoantibodies present in the sample, the sample is contacted with a solid phase to which is bound a binding pair member that will bind to the labeled receptor in an amount related to the amount or presence of autoantibody in the sample. The solid phase is then separated from the liquid phase and in one embodiment, the solid phase is washed with a buffered solution to remove any unbound labeled receptor. After the separation step the presence or amount of autoantibody in the sample is determined by detecting the presence or amount of labeled receptor in either the liquid or solid phases.

The solid phase can be a suspension of particles having affixed thereto an antibody that specifically binds to the labeled receptor. In one embodiment, the label is alkaline phosphatase and labeled receptor is detected by adding a solution containing chemiluminescent substrate to separated solid phase, the substrate being adapted to be activated by the enzyme to generate a detectable signal.

In one embodiment, the method further comprises mixing the sample with an interference blocking reagent prior to or simultaneously with the labeled receptor, the interference blocking reagent being capable of specifically binding to substances that may be present in the sample that bind to the labeled receptor competitively with the autoantibody.

In one aspect, the receptor is intrinsic factor, the autoantibodies are blocking autoantibodies that bind to the B12 binding site on intrinsic factor and the interference blocking reagent is capable of specifically binding to B12 present in the sample. In one aspect that reagent is an antibody that specifically binds B12 and in another aspect the reagent is R-protein that binds B12.

The present invention also provides a test kit for performing assays for autoantibodies. The test kit includes (a) a container containing a labeled receptor, the labeled receptor having a specific binding site for the autoantibody being determined; (b) a container containing a binding pair member capable of binding to the labeled receptor at the binding site for the autoantibody, said binding pair member being bound to a solid phase; and (c) an interference blocking reagent capable of specifically binding to a substance in the sample, the substance being capable of binding to the autoantibody binding site of the labeled receptor.

### DETAILED DESCRIPTION

The invention provides an immunoassay for the detection of autoantibodies in a fluid sample. Autoantibodies that can be detected with the present invention are antibodies to a receptor in a living organism. The presence of the autoantibodies being detected in the organism is typically related to an autoimmune disease. Typically, the autoantibodies will bind to one or more binding sites on a receptor and either block the binding of a substance in the organism that naturally binds to the receptor or when stimulate the release of a hormone. Examples of receptors include intrinsic factor, folate binding protein, insulin, thyroglobulin, thyroid microperoxidase, and thyroid stimulating hormone. The term “protein” is used to herein to include molecules with protein components, polypeptide and peptide fragments.

In a method of the invention a receptor having a binding site to which the autoantibody of interest specifically binds is labeled with a signal generating compound. “Label”, “labeled” and “labeled conjugate” and the like refer to a conjugate of intrinsic factor, antibody, receptor or other binding component with a chemical label such as an enzyme, a fluorescent compound, a radioisotope, a chromophore, or any other detectable chemical specie, the conjugate retaining the capacity to specifically bind to its binding partner. “Detection system,” and the like, as exemplified below, refers to a chemical system that provides detectable signals. Illustrative examples of labels include any of those known in the art, including enzymes, pigments, dyes, fluorophores, radioisotopes, stable free radicals luminescers such as chemiluminescers, biolumiescers, and the like. In a signal detection system useful in the method of the invention, the label is an enzyme the detectable signal of which may be generated by exposing the labeled reagent to a particular substrate and incubating for color, fluorescence or luminescence development.

In one aspect of the invention, the labeled receptor is labeled intrinsic factor wherein the label is desirably an enzyme such as alkaline phosphatase.

The labeled receptor is mixed with the fluid sample suspected of containing autoantibodies for a time sufficient for autoantibodies present in the sample to bind to the labeled receptor. In one aspect of the invention, an interference blocking reagent is mixed with the fluid sample prior to or simultaneously with the labeled receptor. The interference blocking reagent is capable of specifically binding to a substance that may be present in the sample, which substance is capable of binding to the autoantibody binding site of the labeled receptor to block or interfere with the binding of autoantibody to the site. In one aspect of the invention the interference blocking reagent further includes components that bind or react with substances present in the sample that interfere with the performance of the assay. These components include, without limitation, scavenger alkaline phophatase, goat and mouse immunoglobulin (IgG). The scavenger alkaline phophatase will desirably block human antibodies to alkaline phosphatase that may cause interference in an assay such as this where an alkaline phosphatase label is used. Mouse IgG will desirably block human anti-mouse antibodies and goat IgG will desirably block non-specific interference between proteins from various species. Other blocking proteins such as bovine IgG, rabbit IgG, burro IgG (or serum proteins for any of these) may be used. A person of ordinary skill will know how to select components most suited to reduce interference from substances that may be in the sample, which components will vary depending on the type of label, specific binding pair member, receptor or other reagent used in the assay. For example, other blocking proteins such as bovine IgG, rabbit IgG, burro IgG (or serum proteins for any of these) may used. In one aspect, an aggregated monoclonal antibody that blocks human anti-mouse antibodies is included in the interference blocking reagent.

As used herein, “specific binding” and “specifically bound” means that the reagent, substance or autoantibody is a binding pair member that binds or is bound to a desired substance or element with specificity, i.e., has a higher binding affinity and/or specificity to the substance or element than to any other moiety. Such binding pairs are well known and include the following: antigen-antibody, growth factor-receptor, nucleic acid-nucleic acid binding protein, complementary pairs of nucleic acids and the like. Where the binding pair member or reagent is described herein as an antibody, the term “antibody” is intended to encompass an effective portion thereof retaining specific binding activity for the substance or element. Effective portions include, for example Fv, scFv, Fab, Fab₂ and heavy chain variable regions or a chimeric molecule or recombinant molecule or an engineered protein comprising any of the portions. Specific examples of binding pair members include: vitamin B12 and intrinsic factor, vitamin B12 and R-protein, biotin and avidin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence protein, polymeric acids and bases, dyes and protein binders, peptides and specific protein binders (e.g., ribonuclease, S-peptide and ribonuclease S-protein), and the like. Furthermore, specific binding pairs can include members that are analogs of the original specific binding member, for example the cyanocobalamin analog, cobinamide, may bind R-protein.

As used herein, the words “competitively, competitive, and compete” in reference to the binding of substances to a member of binding pair refer to the ability of one such substance in a sample (an analyte) to compete with another substance for same binding site on binding pair member, that is, the two substances have the same binding specificity for that binding pair member.

After the labeled receptor has been mixed with the fluid sample for a sufficient amount of time for at least some of the autoantibodies present in the sample to bind to the receptor, a binding pair member bound to a solid phase is contacted with the mixture, the binding pair member is capable of binding to the labeled receptor in an amount that is related to the presence or amount of autoantibodies to the receptor present in the sample.

As used herein, “bound to” with reference to the solid phase encompasses all mechanisms for binding antibodies and proteins, directly or indirectly to surfaces of solid phases so that when the solid phase is mixed with sample and the binding pair member interacts with its surrounding environment the binding pair member remains associated with the surface. Such mechanisms chemical or biochemical linkage via covalent attachment, attachment via specific biological binding (e.g., biotin/streptavidin), coordinative bonding such as chelate/metal binding, or the like. In one aspect of the invention, the binding pair member is a mouse anti-intrinsic factor antibody bound to the solid phase through an anti-mouse antibody attached to the surface of the solid phase.

“Solid phase” as used herein refers to an insoluble material to which one component of the assay may be bound. Known materials of this type include hydrocarbon polymers such as polystyrene and polypropylene, glass, metals, and gels. Such supports may be in the form of beads, tubes, strips, disks and the like. Magnetic particles are particularly preferred for use with the assays of this invention.

Autoantibodies to a receptor may be of at least two types, blocking and nonblocking. Blocking autoantibodies directly compete with a substance or analyte for a receptor binding site and nonblocking autoantibodies prevent the substance or analyte from binding to a receptor by conformationally changing the receptor or by sterically hindering binding to the binding site. Two types of anti-intrinsic factor antibodies to intrinsic factor have been identified: the blocking antibody (Type I), which blocks the binding of B12 to intrinsic factor and a nonblocking antibody.(Type II) (this antibody is also referred to as the binding antibody), which reacts with the intrinsic factor-B12 complex. In one embodiment of the invention, the binding pair member bound to the solid phase is selected so that if Type II antibody binds to the labeled intrinsic factor, labeled intrinsic factor is hindered from binding to the binding pair member-coated solid phase.

After the binding pair member coated solid phase has been contacted with the sample for a sufficient time and under appropriate conditions to allow labeled receptor to bind to the binding pair member in an amount related to the presence or amount of autoantibodies in the sample, the solid phase is separated from the liquid phase and the presence of labeled receptor in either phase is determined.

In another embodiment, the present invention provides a test kit for performing assays for autoantibodies. The test kit includes (a) a container containing labeled receptor, the labeled receptor having a specific binding site for the autoantibody being determined; (b) a container containing a binding pair member capable of binding to the labeled receptor at the binding site for the autoantibody, said binding pair member being bound to a solid phase; and (c) an interference blocking reagent capable of specifically binding to a substance in the sample, the substance being capable of binding to the autoantibody binding site of the labeled receptor. “Container” as used herein may be one portion of a reagent pack into which separate components may be placed to keep the components from coming into contact with each other. In one aspect of the invention, the kit is adapted for use with an automated immunoassay analyzer, such as the ACCESS® Immunoassay System, the ACCESS®2 Immunoassay System, the UniCel™ DxI 800 Immunoassay System and the Synchron LX®I725 Clinical System (the “Beckman Systems”), each of which is available from Beckman Coulter, Inc. of Fullerton, Calif.

In this embodiment, the test kit is typically a reagent pack comprising a plurality of wells adapted for use with a Beckman System, wherein one reagent well (container) comprises the solid phase which is paramagnetic particles having a binding pair member bound thereto capable of binding to labeled receptor, another reagent well comprises the labeled receptor and another reagent well comprises an interference blocking reagent.

“Kit” is used herein to refer to a combination of reagents usually formulated with necessary buffers, salts, and stabilizers, where the reagents are premeasured so as to at least substantially optimize the assay sensitivity.

One aspect of the invention provides for the use of a test kit to detect the presence or amount of autoantibodies to intrinsic factor using an automated analyzer. In this embodiment, a predetermined amount of interference blocking reagent taken from the reagent well and delivered to a reaction vessel followed by a known quantity of fluid sample and a predetermined amount of labeled receptor taken from the reagent well is added. The mixture is allowed to incubate on the system. After the incubation period, a predetermined amount of the solid phase having a binding pair member bound thereto is aspirated from the reagent well and delivered to the reaction mixture. The solid phase is separated from the liquid phase by pulling the paramagnetic particles out of suspension using magnets. The solid phase is washed to remove any uncaptured reagents and the label detection substrate is added. The signal generated is related to the amount of autoantibody present in the sample.

The following example is illustrative of the invention and is not intended to limit the scope of the invention as set out in the appended claims.

### EXAMPLE I

### Assay for Autoantibodies to Intrinsic Factor (Type I Autoantibodies)

Enzyme labeled intrinsic factor was prepared as described in published Canadian patent application no. 2,110,019, the teachings of which are hereby incorporated by reference. Briefly, maleimide groups were introduced onto alkaline phosphatase (ALP) by reaction with a 3-6 fold molar excess of sulfosuccinimidyl 4-(N-maleimidomethyl)cyclohexane-1-carboxylate in 0.2 M imidazole, pH 9.0; excess reagent was removed by gel filtration on Sephadex G-50 in PBS. Purified intrinsic factor was reacted with 40 mM N-acetyl homocysteine thiolactone in 0.2 M Imidazole and 1.0 mM EDTA, pH 9.0; excess reagent was removed on a second gel filtration column in the PBS with 1 mM EDTA. Conjugation was achieved by incubating modified intrinsic factor with a 6-fold molar excess of modified enzyme for two hours at room temperature, followed by 16 hours at 2-8° C. The conjugate was purified by size exclusion chromatography on Superdex 200 in Tris-buffered saline, pH 8.0.

The solid phase was prepared by binding mouse monoclonal anti-intrinsic factor that binds to the B12 binding site of intrinsic factor to goat anti-mouse immobilized on magnetic particles at the level of 1.5 μg per mg of particles and used as a 1 mg/ml particle suspension stock. Antibody was adsorbed for 2-3 hours in suspension. Afterward, the particles were washed twice and resuspended in a 0.1 M Tris buffer at pH 8.00. (0.1 M Tris, 0.1% BSA, 2.0 mM Mgcl3, 2.0 mM MgCl2, 0.1 mM ZnCl2, 0.15 M NaCl, 0.02% Tween 20, 0.1% Sodium azide, 0.1% ProClin) Purified mouse monoclonal anti-intrinsic factor was obtained as described in Canadian published patent application No. 2,110,019. Other methods of obtaining such monoclonal antibodies are described in U.S. Pat. No. 5,506,109, the teachings of which are hereby incorporated by reference.

In this embodiment, interference blocking reagent was used in the assay for the autoantibodies. The reagent was prepared using purified mouse monoclonal anti-vitamin B12 (Cone CD-29, purchased from Sigma-Aldrich, Co. St. Louis, Mo. USA) in a buffered protein solution. The reagent also included, without limitation, scavenger alkaline phophatase, goat and mouse immunoglobulin (IgG).

The assay to determine the presence or amount of autoantibodies to intrinsic factor was conducted using the ACCESS® Immunoassay Analyzer. 55 microliters of the interference blocking reagent was drawn from reagent well comprising anti-B12 antibody in a concentration of about 4.3 micrograms/milliliter and 50 microliters deposited into a reaction vessel. 50 microliters of sample was then added to the reaction vessel. 50 microliters of intrinsic factor-alkaline phosphatase conjugate was added to the reaction vessel and the mixture was incubated for 20 minutes at about 37° C. 55 microliters of the anti-intrinsic factor coated solid phase were aspirated from a reagent well comprising solid phase at a concentration of about 1.0 milligrams/milliliter. 50 microliters were added to the reaction vessel and the mixture allowed to incubate for five minutes at about 37° C. The solid phase was separated from the liquid phase and washed three times. A chemiluminescent substrate that reacts with alkaline phosphatase to generate a detectable signal (Lumi-Phos® 530, commercially available from Lumigen, Inc., Detroit, Mich.) was then added to the reaction vessel and the signal measured with a luminometer. The assay is semi-quantitative. The results were reported as a ratio of relative light units (RLUs) of total RLUs of a calibrator divided by the sample RLUs. As the amount of autoantibodies to intrinsic factor increase, the signal decreases and results ratio increases. Results may be reported as negative, equivocal or positive for autoantibodies. If a quantitative result is desired a determination of the amount of autoantibodies present in the sample may be determined from a stored calibration. The results are expressed in Antibody Units/milliliter (AU/mL).

The sensitivity and range of the assay for anti-intrinsic factor antibodies performed in accordance with this invention was compared to that of a commercially available RIA obtained from Diagnostics Products Corporation and found to be greater and to allow better discrimination between negative and positive samples.

While embodiments of the present invention have been described, it should be understood that various changes, adaptations and modifications may be made therein without departing from the spirit of the invention and the scope of the appended claims.

1. A method for detecting the presence of an autoantibody comprising:
a) mixing a fluid sample suspected of containing the autoantibody with a labeled receptor, the receptor having a specific binding site for said autoantibody;
b) contacting the mixture from step (a) with a solid phase to which is bound a binding pair member that will bind to the labeled receptor in an amount related to the presence or amount of autoantibody present in the sample;
c) separating the solid phase from the liquid phase;
d) determining the presence or amount of autoantibody by determining the presence or amount of labeled receptor remaining in the liquid phase or bound to the binding pair on the solid phase.

2. The method of claim 1 further comprising mixing an interference blocking reagent that will bind to a substance in the fluid sample, said substance being capable of binding to the autoantibody binding site of the labeled receptor and wherein the interference blocking reagent is mixed with the fluid sample prior to or simultaneously with the labeled receptor.

3. The method of claim 1 wherein the labeled receptor is labeled intrinsic factor and the autoantibody binds to intrinsic factor at a binding site that when the autoantibody is bound thereto will block the binding of vitamin B12 to the intrinsic factor receptor.

4. The method of claim 3 further comprising mixing an interference blocking reagent with the fluid sample prior to or simultaneously with the labeled intrinsic factor, wherein the interference blocking reagent specifically binds to vitamin B12.

5. The method of claim 1 wherein the binding pair member bound to the solid phase is a monoclonal antibody.

6. A method for detecting anti-intrinsic factor autoantibody in a fluid sample comprising:
a) adding labeled intrinsic factor to the fluid sample, the labeled intrinsic having a binding site to which anti-intrinsic autoantibody will bind competitively with any vitamin B12 present in the fluid sample;
b) adding an interference blocking reagent with the fluid sample prior to or simultaneously with the labeled intrinsic factor, the interference blocking reagent being capable of specifically binding to vitamin B12;
c) adding to the mixture of (a) and (b) a solid phase to which is bound a binding pair member that will bind labeled intrinsic factor in an amount related to the presence or amount of autoantibody present in the fluid sample; and
d) determining the presence or amount of autoantibody by determining the presence or amount of labeled intrinsic factor remaining in the liquid phase or bound to the binding pair member on the solid phase.

7. The method of claim 6 wherein the binding pair member is an antibody that will bind to labeled intrinsic factor only when the intrinsic factor is not bound to autoantibodies present in the sample.

8. The method of claim 6 wherein the presence of the labeled intrinsic factor is determined using a detection system.

9. The method of claim 8 wherein the detection system includes the use of a label selected from the group consisting of a radioisotope, an enzyme, a substrate of an enzyme reaction, a fluorescent label and a chemiluminescent label and means for detecting such labels.

10. The method of claim 6 wherein the label is an enzyme.

11. The method of claim 10 wherein the label is alkaline phosphatase.

12. The method of claim 1 1 further comprising separating the solid phase with bound labeled intrinsic factor from the liquid phase and mixing the solid phase with a chemiluminescent substrate.

13. A test kit for performing an assay to detect the presence or amount of autoantibody in a sample, said test kit comprising: (a) a container containing a labeled receptor, the receptor having a specific binding site for said autoantibody; (b) a container containing a binding pair member capable of binding to the labeled receptor at the binding site for the autoantibody, said binding pair member being bound to a solid phase; and (c) an interference blocking reagent that will specifically bind to a substance in the sample, the substance being capable of binding to the autoantibody binding site of the labeled receptor.

14. The test kit of claim 13 wherein said solid phase of (b) are paramagnetic particles.

15. The test kit of claim 13 wherein said label is selected from the group consisting of consisting of an enzyme, a substrate of an enzyme reaction, a fluorescent label and a chemiluminescent label.

16. The test kit of claim 13 wherein the labeled receptor is labeled intrinsic factor and the interference blocking reagent is capable of specifically binding to vitamin B12.

17. The test kit of claim 16 wherein the interference blocking reagent is an antibody to vitamin B12.

18. The test kit of claim 17 wherein the antibody is a monoclonal antibody.

19. The test kit of claim 16 wherein the interference blocking reagent is R-protein.

20. The test kit of claim 16 wherein the label is alkaline phosphatase.

21. The test kit of claim 16 wherein the binding pair member is an anti-intrinsic factor antibody that binds to the vitamin B 12 binding site of intrinsic factor and the solid phase are paramagnetic particles.

22. The test kit of claim 21 wherein the antibody is a monoclonal antibody.
